(19) United States
(12) Patent Application Publication
(10) Pub. No.: US 2004/0146481 A1
(43) Pub. Date: Jul. 29, 2004

(54) Hair care products with natural oils

(76) Inventors: Peter Busch, Erkrath (DE); Wolf Eisfeld, Duesseldorf (DE); Petra Biehl, Neuss (DE); Ulrich Issberner, Rommerskirchen (DE)

Correspondence Address: COGNIS CORPORATION, PATENT DEPARTMENT, 300 BROOKSIDE AVENUE, AMBLER, PA, 19002, US

(21) Appl. No.: 10/479,131

(22) PCT Filed: May 22, 2002
(86) PCT No.: PCT/EP02/05589

(30) Foreign Application Priority Data
May 31, 2001 (DE) 101 26 448.8

(51) Int. Cl. A61K 7/06

(52) U.S. Cl. 424/74

(57) ABSTRACT

A process for inhibiting hair breakage involving contacting hair with a composition containing: (a) a natural oil in solid or semisolid form; and (b) a glyceryl dioleate.

This application is a 371 of PCT/EP02/05589 filed May 22, 2002.

This invention relates generally to cosmetic preparations and, more particularly, to hair care preparations containing semisolid to solid natural oils and glyceryl dioleate which may be used with advantage for the care of dry or damaged hair and for preventing drying out and split ends and for the styling of hair.

### PRIOR ART

Numerous hair care preparations for preventing split ends and hair breakage are already available on the market. Damage by environmental influences, treatment with chemicals or frequent and intensive styling leads to roughening of the hair shaft and is even macroscopically visible after only a short time. In addition, ageing of the hair often leads to dry and brittle hair. To counter these effects, European patent application 0 943 313 discloses the use of sunflower wax and optionally jojojba wax in hair care preparations for counteracting damaging environmental influences. Hair care preparations containing such waxes lead to improved combability and styling behavior. The use of fats in hair care preparations is also known from brilliantines and modern styling preparations. Besides other components, semisolid to solid fat-like components are also required for the production of styling preparations intended in particular for dark, very thick hair. Corresponding fats are described in the patent literature (JP 2000143454 A2). Unfortunately, a disadvantage of conventional styling preparations is that they often dry out after application.

Accordingly, the problem addressed by the present invention was to provide preparations which would have an improved effect against hair breakage and split ends, dryness of the hair and scalp and against environmental influences and hair ageing effects. The effects of processes which damage the hair through increased drying out, such as permanent waving and styling, would also be counteracted. In addition, the preparations according to the invention would cause less damage to the hair after application and would stabilize the hair and thus contribute towards maintaining the hair structure. These preparations would also show high dermatological compatibility and would be distinguished by high stability during storage at elevated temperatures.

### DESCRIPTION OF THE INVENTION

The present invention relates to the use of hair care preparations containing semisolid to solid natural oils and glyceryl dioleate for styling hair and for preventing split ends, increasing luster, caring for dry hair, improving combability and establishing an even water/moisture balance in the hair in different weather conditions.

The present invention also relates to the use of glyceryl dioleate as a lipid layer enhancing component in hair care preparations. The fats used may also be enriched with sterols, tocopherols and/or carotinoids.

It has been found that hair care preparations containing semisolid to solid natural oils and glyceryl dioleate have an improved effect against split ends and dry hair. They are particularly suitable for the styling of hair because the common problem of drying out of the scalp and hair after the application of styling preparations can be lastingly avoided. The glyceryl dioleate has a lipid layer enhancing effect so that corresponding preparations lead—even prophylactically—to reduced damage to the hair structure and thus contribute towards maintaining the hair structure. As a lipid layer enhancer, glyceryl dioleate protects the hair against further drying out and split ends, increases luster and provides for an even water/moisture balance in different weather conditions. In addition, a distinct improvement in the combability of the hair is also observed after application of the hair care preparations according to the invention.

The described hair care preparations are particularly suitable for the treatment of ageing hair and dry hair and for protection against weather conditions which contribute towards increased drying out of the hair.

Depending on the consistency of the oils used and particularly with relatively high percentage contents of solid oils, even thick and dense hair can readily be styled with the formulations according to the invention.

Natural Semisolid to Solid Oils

The preparations according to the invention contain 0.1 to 20% by weight, preferably 0.5 to 10% by weight and more particularly 1.5 to 6.5% by weight of the natural semisolid to solid oils. Natural oils in the context of the invention are understood to be oils of animal and preferably vegetable origin. The natural oils in question are esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are triglycerides based on C₆₋₁₀ fatty acids, liquid mono-/di-/triglyceride mixtures based on C₈₋₁₈ fatty acids. In order to be able to maintain the semisolid to solid consistency, corresponding mixtures of the oils may also be used.

The natural oils or oil mixtures, which are semisolid to solid at room temperature, have a melting range above 20° C. and preferably above 25° C.

They may contain sterols, tocopherol, unsaturated fatty acids and/or carotinoids. Sterols may be present in the oils in quantities of 0.01 to 5% by weight, preferably 0.1 to 3% by weight and more particularly 1 to 2% by weight, tocopherols in quantities of 0.01 to 5% by weight, preferably 0.03 to 3% by weight and more particularly 0.05 to 2% by weight, unsaturated fatty acids in quantities of 0.1 to 90% by weight, preferably 10 to 80% by weight and more particularly 30 to 70% by weight and/or carotinoids in quantities of 0.01 to 2% by weight, preferably 0.03 to 1% by weight and more particularly 0.05 to 0.5% by weight.

The commercial products of Aarhus Oliefabrik A/S, Aarhus, Denmark, known under the name of Cremeol®, now Cegesoft® (Cognis, Düsseldorf), are preferred. They include

Cegesoft® HF-52 (hydrogenated vegetable oil)

Cegesoft® HF-62 (hydrogenated vegetable oil)

Cegesoft® FR-57 (hydrogenated vegetable glycerides)

Cegesoft® (formerly Cremeol®) SH, shorea stenoptera butter, C18:1 35%, C18:2 1%, unsaponifiable matter (triterpenes and sterols) 1.4% (of which 31.5% triterpenes, 4-desmethylsterols 65.3%+α-methylsterols 3.2%)

Cegesoft® VP (mixture of vegetable oil, hydrogenated vegetable oil and candelilla wax)

Cegesoft® (formerly Cremeol®) PS 6, vegetable oil, C18:1 84%, C18:2 5%, unsaponifiable matter (mainly phytosterols, e.g. β-sitosterol, campesterol) 1.1%, tocopherols 1,400 ppm

Cegesoft® (formerly Cremeol®) PS 17, vegetable oil, C18:1 72%, C18:2 11%, unsaponifiable matter (mainly phytosterols, z.B. β-sitosterol, campesterol) 1.5%, tocopherols 2000 ppm

Cegesoft® (formerly Cremeol®) PFO, passionflower (Passiflora incarnata) oil, C18:1 16%, C18:2 70%, unsaponifiable matter (phytosterols, z.B. β-Sitosterol, campesterol)<1−max. 2.5% (of which β-sitosterol 49%, stigmasterol 23%, campesterol 11%, others 17%), tocopherols 600 ppm

Cegesoft® (formerly Cremeol®) GPO, golden palm (Elaeis guineensis) oil, tocopherols+tocotrienols 1000 ppm, carotinoid 400 ppm

Cegesoft® (formerly Cremeol®) SBE, shea butter (Butyrospermum parkii) extract, C18:1 63.4%, C18:2 1.8%, unsaponifiable matter: 30-40%, of which triterpene alcohols 85%, sterols 8%; tocopherols 1,400 ppm

The semisolid to solid oils are present in the preparations according to the invention in combination with glyceryl dioleate Cremeol® FR-36. This component is used as a lipid layer enhancer in the hair care preparations so that it has on the one hand a caring effect and, on the other hand, a protective effect. Glyceryl dioleate is used in the preparations in quantities of 0.1 to 5% by weight, preferably 0.5 to 3% by weight and more particularly 1 to 2% by weight. It acts from surfactant-free and surfactant-containing formulations and is particularly suitable for dry and relatively old hair.

Commercial Applications

In one proven embodiment, the hair care preparations contain 0.1 to 20% by weight semisolid to solid natural oils and 0.1 to 5% by weight glyceryl dioleate. Preparations containing 0.5 to 10% by weight semisolid to solid natural oils and 0.5 to 3% by weight glyceryl dioleate are preferred while hair care preparations containing 1.5 to 6.5% by weight of semisolid to solid natural oils and 1 to 2% by weight glyceryl dioleate are particularly preferred.

The preparations according to the invention are used in the form of hair gels, hair waxes, brilliantines, foams, gloss sprays and special washing preparations for hair tips. Besides preventing hair breakage and split ends, however, the preparations may also be used for intensive care of the scalp, for increasing luster and volume and for improving feel and moisture balance.

Cosmetic and/or Pharmaceutical Preparations

The hair care preparations according to the invention may contain surfactants, co-emulsifiers, superfatting agents, pearlizing waxes, consistency factors, polymers, silicone compounds, waxes, stabilizers, antidandruff agents, film formers, swelling agents, hydrotropes, preservatives, solubilizers, complexing agents, reducing agents, alkalizing agents, antioxidants, perfume oils and the like as additional auxiliaries and additives.

Cationic surfactants in the form of esterquats are frequently used in hair care preparations. “Esterquats” are generally understood to be quaternized fatty acid triethanolamine ester salts. They are known compounds which may be obtained by the relevant methods of preparative organic chemistry, cf. International patent application WO 91/01295 (Henkel), in which triethanolamine is partly esterified with fatty acids in the presence of hypophosphorous acid, air is passed through the reaction mixture and the whole is then quaternized with dimethyl sulfate or ethylene oxide. In addition, DE-C1 4308794 (Henkel) describes a process for the production of solid esterquats in which the quaternization of triethanolamine esters is carried out in the presence of suitable dispersants, preferably fatty alcohols. Overviews of this subject have been published, for example, by R. Puchta et al. in Tens. Surf. Det., 30, 186 (1993), by M. Brock in Tens. Surf. Det., 30, 394 (1993), by R. Lagerman et al. in J. Am. Oil Chem. Soc., 71, 97 (1994) and by I. Shapiro in Cosm. Toil. 109, 77 (1994). The quaternized fatty acid triethanolamine ester salts correspond to formula (I):

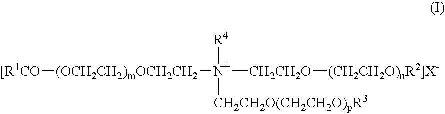

in which R¹CO is an acyl group containing 6 to 22 carbon atoms, R² and R³ independently of one another represent hydrogen or have the same meaning as R¹CO, R⁴ is an alkyl group containing 1 to 4 carbon atoms or a (CH₂CH₂O)_{q}H group, m, n and p together stand for 0 or numbers of 1 to 12, q is a number of 1 to 12 and X is halide, alkyl sulfate or alkyl phosphate. Typical examples of esterquats which may be used in accordance with the present invention are products based on caproic acid, caprylic acid, capric acid, lauric acid, myristic acid, palmitic acid, isostearic acid, stearic acid, oleic acid, elaidic acid, arachic acid, behenic acid and erucic acid and the technical mixtures thereof obtained, for example, in the pressure hydrolysis of natural fats and oils. Technical C_{12/18} cocofatty acids and, in particular, partly hydrogenated C_{16/18} tallow or palm oil fatty acids and C_{16/18} fatty acid cuts rich in elaidic acid are preferably used. To produce the quaternized esters, the fatty acids and the triethanolamine may be used in a molar ratio of 1.1:1 to 3:1. With the performance properties of the esterquats in mind, a ratio of 1.2:1 to 2.2:1 and preferably 1.5:1 to 1.9:1 has proved to be particularly advantageous. The preferred esterquats are technical mixtures of mono-, di- and triesters with an average degree of esterification of 1.5 to 1.9 and are derived from technical C_{16/18} tallow or palm oil fatty acid (iodine value 0 to 40). In performance terms, quaternized fatty acid triethanol-amine ester salts corresponding to formula (I), in which R¹CO is an acyl group containing 16 to 18 carbon atoms, R² has the same meaning as R¹CO, R³ is hydrogen, R⁴ is a methyl group, m, n and p stand for 0 and X stands for methyl sulfate, have proved to be particularly advantageous. Besides the quaternized fatty acid triethanolamine ester salts, other suitable esterquats are quaternized ester salts of fatty acids with diethanol-alkyamines corresponding to formula (II):

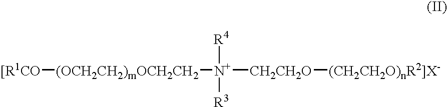

in which R¹CO is an acyl group containing 6 to 22 carbon atoms, R² is hydrogen or has the same meaning as R¹CO, R³ and R⁴ independently of one another are alkyl groups containing 1 to 4 carbon atoms, m and n together stand for 0 or numbers of 1 to 12 and X stands for halide, alkyl sulfate or alkyl phosphate. Finally, another group of suitable esterquats are the quaternized ester salts of fatty acids with 1,2-dihydroxypropyl dialkylamines corresponding to formula (III):

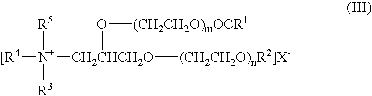

in which R¹CO is an acyl group containing 6 to 22 carbon atoms, R² is hydrogen or has the same meaning as R¹CO, R³, R⁴ and R⁵ independently of one another are alkyl groups containing 1 to 4 carbon atoms, m and n together stand for 0 or numbers of 1 to 12 and X stands for halide, alkyl sulfate or alkyl phosphate. So far as the choice of the preferred fatty acids and the optimal degree of esterification are concerned, the examples mentioned for (I) also apply to the esterquats corresponding to formulae (II) and (III). The esterquats are normally marketed in the form of 50 to 90% by weight solutions in alcohol which may readily be diluted with water as required. Esterquats may be present in the hair care preparations in quantities of 0 to 10% by weight, preferably 1 to 5% by weight and more particularly 1.5 to 3% by weight.

Other preferred auxiliaries and additives are anionic and/or amphoteric or zwitterionic surfactants. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl-(ether) phosphates. If the anionic surfactants contain polyglycol ether chains, they may have a conventional homolog distribution although they preferably have a narrow-range homolog distribution. Typical examples of amphoteric or zwitterionic surfactants are alkylbetaines, alkylamido-betaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. The percentage content of surfactants in the preparations may be from 0.1 to 10% by weight and is preferably from 0.5 to 5% by weight, based on the preparation.

Other surfactants may also be added to the hair care preparations as co-emulsifiers, including for example

(1) products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;

(2) C_{12/18} fatty acid monoesters and diesters of addition products of 1 to 30 mol ethylene oxide onto glycerol;

(3) sorbitan mono- and diesters of saturated and unsaturated fatty acids containing 6 to 22 carbon atoms and ethylene oxide addition products thereof;

(4) alkyl mono- and oligoglycosides containing 8 to 22 carbon atoms in the alkyl group and ethoxylated analogs thereof;

(5) addition products of 15 to 60 mol ethylene oxide onto castor oil and/or hydrogenated castor oil;

(6) polyol esters and, in particular, polyglycerol esters such as, for example, polyglycerol polyricinoleate, polyglycerol poly-12-hydroxy-stearate or polyglycerol dimerate isostearate. Mixtures of compounds from several of these classes are also suitable;

(7) addition products of 2 to 15 mol ethylene oxide onto castor oil and/or hydrogenated castor oil;

(8) partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);

(9) mono-, di and trialkyl phosphates and mono-, di- and/or tri-PEG-alkyl phosphates and salts thereof;

(10) wool wax alcohols;

(11) polysiloxane/polyalkyl polyether copolymers and corresponding derivatives;

(12) mixed esters of pentaerythritol, fatty acids, citric acid and fatty alcohol according to DE 1165574 PS, polyalkylene glycols and

(13) glycerol carbonate

The addition products of ethylene oxide and/or propylene oxide onto fatty alcohols, fatty acids, alkylphenols, sorbitan mono- and diesters of fatty acids or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations from DE 2024051 PS.

C_{8/18} alkyl mono- and oligoglycosides, their production and their use as surfactants are known from the prior-art literature. They are produced in particular by reacting glucose or oligosaccharides with primary alcohols containing 8 to 18 carbon atoms. So far as the glycoside component is concerned, both monoglycosides where a cyclic sugar unit is attached to the fatty alcohol by a glycoside bond and oligomeric glycosides with a degree of oligomerization of preferably up to about 8 are suitable. The degree of oligomerization is a statistical mean value on which a homolog distribution typical of such technical products is based.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine.

Superfatting agents may be selected from such substances as, for example, lanolin and lecithin and also polyethoxylated or acylated lanolin and lecithin derivatives, polyol fatty acid esters and fatty acid alkanolamides, the fatty acid alkanolamides also serving as foam stabilizers.

The consistency factors mainly used are fatty alcohols or hydroxyfatty alcohols containing 12 to 22 and preferably 16 to 18 carbon atoms and also fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are polymeric thickeners, such as Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens®) [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat poly-peptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Besides the natural oils used, waxes may also be present in the preparations, more especially natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Metal salts of fatty acids such as, for example, magnesium, aluminium and/or zinc stearate or ricinoleate may be used as stabilizers.

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The complexing agents used may be selected from EDTA, NTA, phosphonic acids, Triton B, turpinal and phenacetin. In addition, reducing agents such as, for example, ascorbic acid, sodium sulfate, sodium thiosulfate and the like may be present. Suitable alkalizing agents are ammonia, monoethanolamines, (L) arginine, AMP, etc.

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

### FORMULATION EXAMPLES

The Cremeol types were melted and dissolved with stirring in Plantacare® 818 UP. All other surfactants were then added and homogenized by stirring. Finally, water and Carbopol® EDT were added and the pH was adjusted to 6.0-6.8 by addition of NaOH with slow stirring.

TABLE 1
Hair shampoo formulations (quantities in % by weight)

| 2-in-1 Shampoo | | | | | | |
|---|---|---|---|---|---|---|
| Sodium Laureth Sulfate | 12 | 12 | 12 | 12 | 12 | 12 |
| TEXAPON ® N 70, | | | | | | |
| Cognis Dusseldorf | | | | | | |
| Cocamidopropyl Betaine | 2.5 | 2.5 | 2.5 | 2.5 | 2.5 | 2.5 |
| DEHYTON ® PK 45, | | | | | | |
| Cognis Dusseldorf | | | | | | |
| Coco Glucoside | 8.0 | 8.0 | 8.0 | 8.0 | 8.0 | 8.0 |
| PLANTACARE ® 818 UP | | | | | | |
| Cremeol ® FR-36* | 1.5 | 1.5 | 1.5 | 1.5 | 1.5 | 1.5 |
| Glyceryldioleate | | | | | | |
| PEG-3 Distearate (and) | 5 | 5 | 5 | 5 | 5 | 5 |
| Sodium Laureth Sulfate | | | | | | |
| EUPERLAN ® PK | | | | | | |
| 900 BENZ, Cognis, | | | | | | |
| Dusseldorf | | | | | | |
| Carbomer | 0.3 | 0.3 | 0.4 | 0.4 | 0.3 | 0.3 |
| CARBOPOL ® EDT 2001, | | | | | | |
| BF Goodrich | | | | | | |
| Cremeol ® PS-6 | 1.0 | | | | | |
| Cremeol ® PS-17 | | 1.0 | | | | |
| Cremeol ® PFO | | | 1.0 | | | |
| Cremeol ® GPO | | | | 1.0 | | |
| Cremeol ® SH | | | | | 1.0 | |
| Cremeol ® SBE | | | | | | 1.0 |

| | |
|---|---|
| Water | to 100 |
| Preservative | q.s. |

*Cremeol types were obtained from Aarhus Oliefabrik A/S. Aarhus, DK

To produce the hair masks, the oil phase containing the liquid and solid components, including Dehyquart F 75, was melted at 75 to 80° C. The water phase—likewise heated to 75 to 80° C.—was poured into the hot oil phase and the whole was slowly cooled with stirring to 30° C.

1. Hair care preparations containing semisolid to solid natural oils and glyceryl dioleates.

2. Hair care preparations as claimed in claim 1, characterized in that the semisolid to solid natural oils have a melting range above 20° C.

3. Hair care preparations as claimed in claims 1 and/or 2, characterized in that they contain semisolid to solid natural oils in quantities of 0.1 to 20% by weight.

4. Hair care preparations as claimed in at least one of claims 1 to 3, characterized in that they contain glyceryl dioleate in quantities of 0.1 to 5% by weight.

5. Hair care preparations as claimed in at least one of claims 1 to 4, characterized in that the natural oils contain sterols and/or unsaturated fatty acids.

6. Hair care preparations as claimed in at least one of claims 1 to 5, characterized in that the natural oils contain carotinoids.

7. Hair care preparations as claimed in at least one of claims 1 to 6, characterized in that the natural oils are present in hydrogenated form.

8. The use of the hair care preparations claimed in claims 1 to 7 for styling hair.

9. The use of the hair care preparations claimed in claims 1 to 7 for preventing split ends and dry hair and for treating split ends and dry hair.

10. The use of the hair care preparations claimed in claims 1 to 7 for increasing the luster of hair.

11. The use of the hair care preparations claimed in claims 1 to 7 for establishing an even water/moisture balance of the hair in different weather conditions.

12. The use of glyceryl dioleate as a lipid layer enhancer in hair care preparations.
